(19) United States
(12) Patent Application Publication
(10) Pub. No.: US 2022/0287805 A1
(43) Pub. Date: Sep. 15, 2022

(54) DENTAL IMPLANT AND SET

(71) Applicants: Peter RANDELZHOFER, Muenchen (DE); Stephan STUR, Muenchen (DE)

(72) Inventors: Peter RANDELZHOFER, Muenchen (DE); Stephan STUR, Muenchen (DE)

(21) Appl. No.: 17/618,417

(22) PCT Filed: Jun. 12, 2020
(86) PCT No.: PCT/EP2020/066359

§ 371 Date: Dec. 10, 2021

(30) Foreign Application Priority Data
Jun. 14, 2019 (DE) 10 2019 116 244.0
Aug. 5, 2019 (DE) 10 2019 121 083.6

(51) Int. Cl. A61C 8/02; A61C 8/00; A61F 2/28

(52) U.S. Cl. CPC A61C 8/0006; A61C 8/0043; A61F 2/2846; A61C 8/0012; A61F 2002/285; A61F 2002/2889

(57) ABSTRACT

The present invention relates to a dental implant for supporting, in particular by using a support structure, a cover element. The dental implant comprises a connection element having first and second end sections for connecting the dental implant to an implantation site of a jawbone and a first support element for supporting a cover element of at least a section of the implantation site after implanting the dental implant. The first end section is designed as, or comprises, a first connection section. The first support element comprises a second connection section. The first and second connection sections may be interconnectable. The second end section is designed as, or comprises, an implantation section for the temporary implantation of the connection element on or in the implantation site.

### FIELD OF THE INVENTION

The present invention relates to a dental implant, and to a dental surgical tool for the dentist or oral surgeon.

### BACKGROUND OF THE INVENTION

An implantable dental prosthesis is usually an implant that can be inserted into the jawbone and may also be referred to as an artificial tooth root. The implantable dental prosthesis is thus a basis for a dental replacement to be built on top of it. A connecting part, known as an abutment, is usually first screwed into the implantable dental prosthesis and then the visible dental crown is placed on the connecting part as a prosthetic restoration.

In order to insert the implantable dental prosthesis into the jawbone, a stable and sufficiently large jawbone area is necessary for permanent anchoring of the implantable dental prosthesis. However, this is often not the case, for example because the jawbone has receded after extraction of a natural tooth and a wound healing phase, or because the jawbone is delicately formed depending on the individual patient. This is often referred to as a bone defect or a bone defect site. In such cases, it may be necessary to “build up” the jawbone before implanting the implantable dental prosthesis. A bone regeneration material made from a synthetic bone substitute material, for example hydroxyapatite, and/or from the body's own bone substitute material of the patient can be used for regeneration.

During the bone regeneration phase, the area of the jawbone to which the bone regeneration material has been applied should be demarcated to prevent unwanted ingrowth of surrounding mucosal tissue or other soft tissue, so that the bone regeneration material may grow undisturbed through the bone, starting from the jawbone. For this purpose, the bone regeneration material is regularly covered with a so-called cover membrane, which should prevent the bone regeneration material from being grown through by a non-bone material, for example surrounding mucosal tissue.

The cover membrane is usually made of a polymer, for example polytetrafluoroethylene, abbreviated in PTFE. The cover membrane may be referred to as a cover foil. The temporary attachment of the cover membrane to the jawbone may be carried out by using suitable fasteners, for example using nails.

In order to ensure osseous growth into the bone regeneration material as undisturbed as possible, it is advantageous if the cover membrane provides a cavity or sufficient space for bone growth. This allows bone regeneration to take place inside the cavity without external disturbances, such as mechanical pressure from the surrounding tissue. For this purpose, the cover membrane may be designed to be for example rigid and/or dimensionally stable.

### SUMMARY OF THE INVENTION

It may be an object of the present invention to specify a dental implant (alternative designation: bone shield; the terms dental implant and bone shield are therefore interchangeable herein) which is provided and designed for support, in particular by a support structure such as a support element, a cover membrane. Furthermore, it is an object of the present invention to suggest a set with a dental implant.

The object according to the invention is achieved by the dental implant with features as described herein, the set with the features described herein, and the tool with the features described herein.

For this purpose, the present invention proposes a dental implant.

The dental implant according to the present invention comprises at least one connection element and at least one first support element. The connection element comprises a first end second. It further comprises a second end section for connecting the dental implant in or to an implantation site of a jawbone. The first support element is designed and provided for retaining or supporting a cover element after the dental implant according to the present invention has been implanted at the implantation site in the jawbone. The cover element is designed and provided in order to cover at least a part or section of the augmentation site, the implantation site, the implantation region or an adjacent area.

The first end section is designed as, or comprises, a first connection section.

The first support element comprises a second connection section.

The first connection section and the second connection section are designed to be interconnectable or connectable to each other.

The second end section is designed as, or comprises, an implantation section for temporarily implanting the connection element on or in the implantation site of the jawbone.

Further, the present invention proposes a set encompassing a dental implant according to the present invention. The set further comprises at least one implantable dental prosthesis for remain permanently at the implantation site, implant region or augmentation site, preferably where the dental implant was previously temporarily implanted, and/or comprises an optional further support element which may have one, more or all of the features of the first support element disclosed herein, and/or further comprises a tool for inserting the connection element into the bone. The first support element and the optional second support element differ in at least one geometric feature. The distinguishing geometric feature may include the shape, a dimension, the surface, and/or the arrangement of support structure elements.

The tool according to the present invention is used to introduce a connection element of a dental implant according to the present invention. The tool comprises a gap section for receiving an end region of the connection element.

Embodiments according to the present invention may comprise one or several of the aforementioned and/or following features in any combination, unless the person skilled in the art recognizes the specific embodiment as technically impossible.

In all of the following statements, the use of the expression “may be” or “may have” and so on, is to be understood synonymously with “preferably is” or “preferably has,” and so on respectively, and is intended to illustrate an embodiment according to the present invention.

Whenever numerical words are mentioned herein, the person skilled in the art shall recognize or understand them as indications of numerical lower limits. Unless it leads the person skilled in the art to an evident contradiction, the person skilled in the art shall comprehend for example the specification of “one” (or “a”/“an”) as encompassing “at least one”. This understanding is also equally encompassed by the present invention as the interpretation that a numerical word, for example, “one” (or “a”/“an”) may alternatively mean “exactly one”, wherever this is evidently technically possible for the person skilled in the art. Both understandings are encompassed by the present invention and apply to all numerical words used herein.

Advantageous developments of the present invention are also described herein.

Whenever an embodiment is mentioned herein, it is then an exemplary embodiment according to the present invention.

The dental implant according to the present invention is preferably not an implantable dental prosthesis which is intended and/or suitable to remain permanently or to replace a tooth. It is preferably not intended to support a dental crown.

An implantable dental prosthesis may be referred to as an artificial tooth root, as a reception for the tooth crown, and/or as a screw. An implantable dental prosthesis may be a base for a dental replacement to be built on top of it. A connecting part, often referred to as an abutment, may be screwed into the implantable dental prosthesis. A visible dental crown may then be placed on the connecting part as a prosthetic restoration.

In several embodiments, the dental implant is not designed and/or prepared to be fastened or fixed and/or to be connected or attached on or to an implantable dental prosthesis.

In several embodiments, the dental implant is prepared and/or designed to support a cover membrane. The cover membrane may temporarily cover a bone defect site with a bone regeneration material in order to prepare a bone regeneration at the jawbone for a later implantation of an implantable dental prosthesis. The cover membrane is supposed to support an unhindered bone regeneration and to reduce or prevent undesired ingrowth of surrounding soft tissue. During this bone regeneration phase, the cover membrane may be fixed to the jawbone, for example via pins, nails, screws or a in different way.

The exemplary fixation agents mentioned may be absorbable or non-absorbable.

Optionally, the cover membrane is not fixed to the jawbone.

The cover membrane may, for example, be placed on the support element of the dental implant according to the present invention on the front side towards the oral cavity (in the implanted state). Fixation or adhesion of the cover membrane to the support element may be achieved, for example, by blood from the surgical site of the jawbone.

The cover membrane can be a flexible film. The cover membrane may be a membrane, cover sheet, film, or other cover element.

The cover membrane may be made of an absorbable material or a non-absorbable material.

An absorbable material, as used herein, may be referred to as a self-dissolving material because it is biodegraded and thus quasi dissolved. Purely by way of example, an absorbable material may be collagen or may comprise collagen.

A non-absorbable material, as used herein, may be a plastic, particularly a biocompatible plastic, for example polytetrafluoroethylene (PTFE).

After the bone regeneration phase, which may take several weeks or months, the cover element or cover membrane is either resorbed or surgically removed. The implantable dental prosthesis, which is not the dental implant according to the present invention, may then be placed in the jawbone and in the regenerated bone. The aim of bone regeneration with the cover membrane is usually an increased stability and thus an improved anchoring of the implantable dental prosthesis in the jawbone, which has been enlarged or augmented through bone regeneration.

The dental implant according to the present invention may in several embodiments according to the present invention be inserted into the jawbone, and is provided for the purpose of being implanted before the implantable dental prosthesis is inserted into the jawbone at the same implantation site, implantation region or augmentation site.

For the most undisturbed, osseous growth of the bone regeneration material between the jawbone and the cover membrane, the dental implant according to the present invention may form or generate a cavity by using a support structure. This cavity is in particular geometrically limited by the jawbone with the overlying bone regeneration material and the support structure with the cover membrane.

For example, the dental implant according to the present invention may have an umbrella-shaped structure, with the umbrella pole corresponding to the connection element, the struts in the umbrella for fastening the umbrella cover corresponding to the support element, and the umbrella cover corresponding to the cover membrane.

In some embodiments, the dental implant according to the present invention is a temporary implant for the jawbone. The dental implant may be, or may remain, implanted e.g. for a few weeks or a few months. If the dental implant is made of an absorbable material, usually no further surgery is then required to remove it; the dental implant rather absorbs by itself. If the dental implant was made of a non-absorbable material, it is then removed or explanted from the jawbone in further surgery. Subsequently, the implantable dental prosthesis may be implanted in the implantation site or region, etc., in which the dental implant according to the present invention was initially implanted.

In contrast to the dental implant in some embodiments, an implantable dental prosthesis which may, in addition to the dental implant, optionally be part of the set according to the present invention, is designed to remain in the jawbone as long as possible. For example, an implantable dental prosthesis may remain in the jawbone for at least ten years. In some cases, the implantable dental prosthesis may remain in the jawbone for twenty years, thirty years, or longer. It may support a dental crown or be prepared for this purpose.

The connection element may have an elongated shape with an internally hollow or non-hollow cross-section. The cross-section perpendicular to the longitudinal alignment may be round, oval, angular or otherwise designed. The second end section of the connection element for connecting the dental implant to the implantation site of the jawbone may be pointed, similar to a nail shape. This shape of the second end section may allow easy fastening of the connection element, as it may be introduced into the jawbone by pushing or driving it in.

The connection element or its second end section may have a thread, in particular an external thread, for screwing or screwing-in the connection element, the second end section or at least a region of the second end section, into an implantation site of the jawbone. In other embodiments, the connection element and/or its second end section does not comprise a thread and/or is not intended to be screwed into the jawbone.

The connection element may comprise slits extending in its longitudinal direction.

The outer diameter of a round connection element may be purely exemplarily between 0.5 mm and 2 mm, for example 0.8 mm, 0.9 mm or 1 mm. The overall length of the connection element may purely exemplarily be between 5 mm and 20 mm, for example a shaft length of 10 mm, a length of the first end section of approx. 0.3 mm and a length of the second end section of approx. 3 mm.

The first support element may be designed as a grid element and/or a stiffening element.

In several embodiments, the first support element comprises a grid-shaped structure or a grid-shaped section.

A grid-shaped structure or a grid-shaped section may have, for example, a rectangular outer contour with longitudinal struts and cross struts arranged on the inside. For example, the grid element may have a central longitudinal strut and three, five or more cross struts.

These or differently shaped or arranged internal struts may be arranged at right angles or at a different angle to each other.

The inner struts may be attached to an outer, closed edge. Purely exemplarily, the rectangular outer contour may have a length of approx. 10 mm and a width of approx. 5 mm.

The rectangular outer contour may have rounded corners.

The grid-like structure may alternatively have (for example in top view) a round, oval or other shape as outer contour. A round shape may have an outer diameter of approx. 5 mm, purely exemplarily. A round shape of the grid element may comprise radial struts or struts arranged in a star shape for example three, four, five or more struts. The star-shaped struts may be arranged regularly or irregularly with respect to the angular spacing between them.

In some embodiments, the second connection section is arranged centrally in the middle within the first support element or connected to it.

The first support element can be manufactured as an injection molded element.

In several embodiments, the first connection section of the connection element and the second connection section of the support element correspond to each other (e.g., as parts of a click system, a clamping system, a tongue and groove, a screw and nut, etc.) and/or are matched or coordinated or adjusted to each other for mutual connection.

The first or the second connection section is preferably designed as an insertion opening, wherein the other of these two connection sections is designed as an insertion pin.

For example, the first connection section of the connection element, which corresponds to the first end section of the connection element, is designed as an insertion pin. In this case, the second connection section of the support element is designed as an insertion opening.

The connection between the insertion pin and the insertion opening may be designed, for example, as a transition fit or as a press fit to prevent an independent and unintentional detachment of the connection element from the support element.

The insertion pin and the insertion opening may have the same diameter along their length. Alternatively, the insertion pin may taper towards the tip and the insertion opening may have a correspondingly conical shape. This may facilitate an insertion of the two parts into each other.

In some embodiments, the implantation section is designed as a pointed or tapered or tapering section. This may make it easier to fasten the connection element in the jawbone by driving or pushing it in.

In several embodiments, the first connection section and the second connection section are designed for releasable connection to each other. This enables a modular construction of the connection element with the support element, so that different embodiments can be combined with each other. For example, depending on the shape and size of the bone regeneration on the jawbone, it may be necessary to select a long or short, a thick or thin connection element. Depending on the desired or possible or available surface area of the bone regeneration, it may be advantageous to select appropriately sized support elements and combine them with a selected connection element.

In some embodiments, in the intended implantation state of the dental implant, the first support element has an upper surface facing the oral cavity of the patient and a lower surface facing the implantation site. The second connection section is preferably designed to receive the first connection section at its lower surface. The cover membrane may rest on the top surface of the first support element. Mechanical attachment of the cover membrane, for example to the first support element and/or to the jawbone, is often not necessary. Instead, bonding by blood from the surgical environment of the jawbone may be sufficient for adhesion of the cover membrane to or on the support element.

Depending on the individual situation, the cover membrane may also be anchored to the jawbone.

Anchoring may be performed, for example, by using one or more absorbable or non-absorbable anchoring pins or nails in the jawbone.

An absorbable anchoring pin may be made of, or comprise, for example magnesium, and a non-absorbable anchoring pin may be made of, or comprise, for example titanium or a titanium alloy.

In some embodiments, the connection element does not comprise a threaded section and/or does not comprise a screw head.

In some embodiments, the connection element and/or the first support element is made of, or comprises, a metal, a plastic, and/or a composite material. In particular, said materials are biocompatible.

In some embodiments, the connection element and/or the first support element is made of, or comprises, an absorbable material.

The connection element may be made of e.g. an absorbable magnesium or magnesium-containing material or may comprise same as is described, for example, in patent documents EP 2 744 531 B1 and EP 2 744 532 B1, the scope of which is also made the subject-matter of the present disclosure by reference.

The support element may be made of or comprise a polylactide, purely by way of example.

In several embodiments, the first connection section comprises a conical region onto which the support element may be plugged. Depending on the material pairing used for the connection element on the one hand and the support element on the other hand, the cone angle α may be selected such that a self-locking taper press connection is established between the connection element and the support element.

The tool for driving the connection element into the jawbone may have a circumferential gap section which may serve to receive a tubular or annular end region of the support element, in particular its first connection section or a section or end section thereof.

The gap section may be delimited at its outer boundary and/or its inner boundary by cylindrical structures of different heights.

The height of the gap may be greater than the length of a cone of the connection element.

As a tool for the dentist or oral surgeon, the tool may have a length of less than 3 cm, less than 5 cm or less than 10 cm. Regardless of its length, it may have a width or a diameter of less than 2 cm, less than 1 cm or less than 0.5 cm.

In several embodiments, the support element comprises a central region having an opening for the insertion or passage of an end section of the connection element.

The support element may have a number of, for example, radially outwardly extending or aligned struts or webs between an inner, preferably disc-shaped, region and, for example, an outer ring or, more generally formulated, outer structure of the support element.

For fastening the connection element to the support element, one or more pins may preferably be provided on the connection element, optionally as sole or only connecting structures. They may extend with their ends from the connection element and be bendable.

The support element may be manufactured, for example, from a disc-shaped or tubular semi-finished product as the starting material, which is processed by laser cutting.

For exact positioning of the support element on the connection element during the assembly step, the inner disc of the support element may comprise notches for guiding the pins, they may run in the axial direction, i.e. perpendicular to the radial direction in which the struts extend.

The connection element or its optional inner disc may have an opening, preferably central. This opening may be dimensioned to be placed with its edge on an optionally provided rim or shoulder. An end-face design of the connection element may thus protrude into or through the opening. The opening may be designed as a blind opening or as a through-opening.

One or several of the advantages mentioned above as well as in the following may be achieved by the present invention.

The dental implant according to the present invention may be made entirely or partially of absorbable materials. Thus, advantageously, a surgical or, respectively, operative second intervention may be avoided, which would be necessary if non-absorbable metals or plastics would have to be removed. A surgical intervention on the jawbone is usually a considerable burden and a possible risk, for example in the form of a risk of infection, for the patient. In addition, a surgical intervention is usually associated with considerable costs.

If the cover membrane and/or possible fastening devices of the cover membrane on the jawbone are also made likewise from absorbable materials, this advantage may be increased even further.

The, preferably modular, two-part (or optionally at least two-part or multi-part) dental implant according to the present invention having the connection element as the first component and the support element as the second component advantageously allows the combination of different connection elements and supporting elements. Depending on the patient-specific and anatomical situation, it may be necessary to deviate from the selected sizes and models of the connection element and/or the support element during an operative intervention. In this way, an optimal combination may be advantageously selected, which can be decisive for a later success of a permanent implantation of an implantable dental prosthesis (as an artificial tooth root for the abutment and the crown, i.e. the prosthetic restoration). Thus, extensive or grave subsequent complications, associated with a high burden for the patient and considerable costs, may be advantageously avoided or at least reduced.

The dental implant according to the present invention is an advantageously simple and cost-effective alternative to patient-specific fabrications, as they are currently possible and applied by using imaging procedures and subsequent generative manufacturing methods due to anatomical conditions.

The connection element according to the present invention enables advantageously simple attachment of the dental implant in the jawbone. A complex screw connection in the jawbone may thus be avoided.

### BRIEF DESCRIPTION OF THE DRAWINGS

The present invention is exemplarily explained based on the accompanying, partially simplified, figures in which identical reference numerals denote same or similar components. The following applies in the figures:

FIG. 1 shows a bone regeneration on the jawbone for preparation of the implantation of two implantable dental prosthesis;

FIG. 2a shows a first support element of a dental implant according to the present invention;

FIG. 2b shows a second support element of a dental implant according to the present invention;

FIG. 2c shows a third support element of a dental implant according to the present invention;

FIG. 2d shows a support element of a dental implant according to the present invention in a side view;

FIG. 3a shows a connection element of a dental implant according to the present invention;

FIG. 3b shows a further connection element of a dental implant according to the present invention;

FIG. 3c-k show further embodiments of the connection element;

FIG. 4 shows a dental implant according to the present invention with support element and connection element;

FIG. 4a shows a further embodiment of a dental implant according to the present invention;

FIG. 5 shows the bone regeneration on the jawbone of FIG. 1 with a dental implant according to the present invention;

FIG. 6a-c show a tool for driving the connection element into the jawbone; and

FIG. 7 shows a manufacturing variant for the support element.

### DETAILED DESCRIPTION

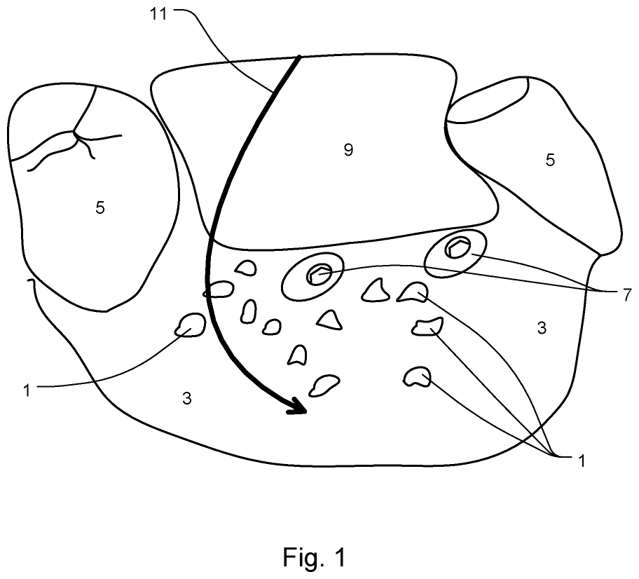

FIG. 1 shows a bone regeneration using bone replacement material 1 on the jawbone 3 for the preparation of the implantation of two implantable dental prosthesis. The bone replacement material 1 is exemplarily designed as granules. The optionally granulated bone replacement material 1 can be synthetic bone replacement material 1, for example hydroxyapatite, and/or natural, endogenous bone replacement material 1. Often these two forms, i.e. synthetic and endogenous material, are mixed together.

The bone replacement material 1 is exemplarily placed and distributed on or over the jawbone 3. The arrangement is shown purely exemplarily and roughly schematically. In practice, the granules are actually often arranged much more densely and also layered on top of each other. The aim of this layered arrangement of bone replacement material 1 is osseous growth, starting from the jawbone 3. Once this bone growth is complete, which usually takes several months, the jawbone, together with the newly formed bone, may form the bony base for implanting an implantable dental prosthesis. The implantable dental prosthesis, which may be described as an artificial tooth root, is then the basis for further reconstruction of a so-called “abutment” and an adjoining dental crown. Based on this description, it is clear that a stable and sufficient bone bed for the implantable dental prosthesis forms the basis for a lasting success, i.e. for the implantable dental prosthesis to remain in the bone for as long as possible.

FIG. 1 also shows two intact teeth 5, between which optional two screws 7 are screwed into the jawbone 3 as placeholders for the subsequent implantable dental prosthesis.

Furthermore, an exemplary cover membrane 9 is shown, which is placed on the granules 1 in the direction of the arrow 11. The cover membrane 9, which may be referred to or designed as a covering film or a covering element, has the function of protecting the bone replacement material 1 against undesired growth of soft tissue, for example of the surrounding mucous membrane, during the osseous growth. Such an ingrowth of soft tissue would significantly limit the stability and strength of the bony structure and should therefore be prevented using the cover membrane 9. However, direct contact of the cover membrane 9 with the granules 1 is disadvantageous to osseous growth, since the pressure of the cover membrane 9 has a disturbing and disadvantageous effect on the growth.

The cover membrane 9 may be affixed to the jawbone 3 for example by using fastening pins on the edge of the cover membrane 9. Nails, screws or the like, which can be absorbable or non-absorbable, may be used as fastening pins.

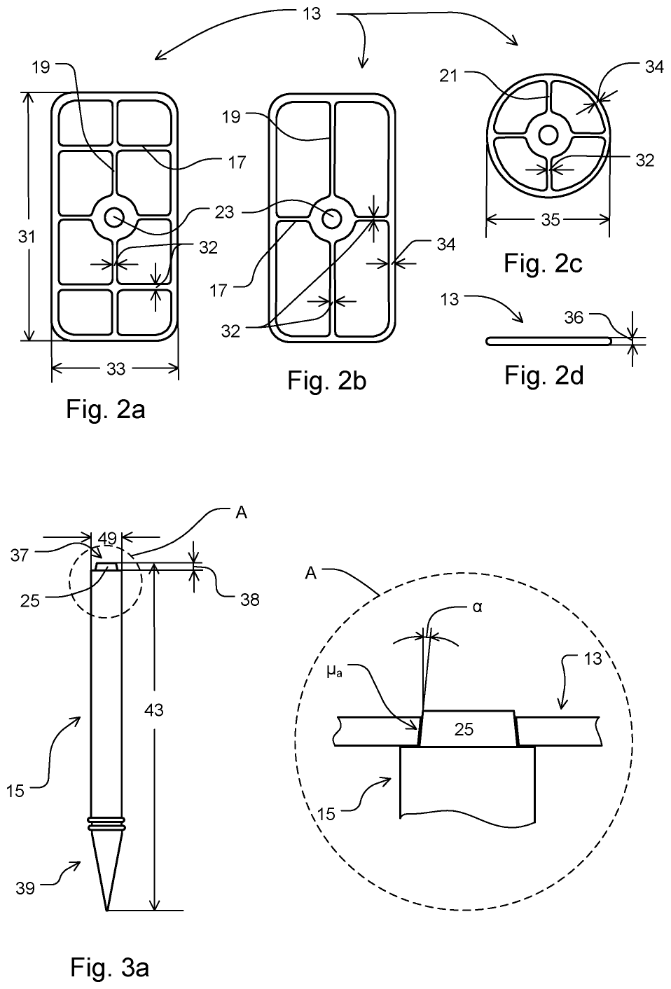

FIGS. 2a, 2b, 2c show examples of differently designed support elements 13 of a dental implant 100 according to the present invention.

The support elements 13 serve to support the cover membrane 9 in order to form, together with the connection element 15, a cavity between the cover membrane 9 and the bone replacement material 1. Osseous growth may take place undisturbed in this cavity. The cover membrane 9 is placed on the top surface of the support element 13, wherein the top surface is the side of the support element 13 facing the oral cavity. In contrast, the lower surface of the support element 13 is oriented toward the jawbone 3. After placement, the cover membrane 9 is affixed or adhered to the support element 13 optionally by blood or the fibrin contained therein. Further optional fixing of the cover membrane 13 to the jawbone 3 is usually not necessary.

The different geometric shapes of the various support elements 13 in FIG. 2a, FIG. 2b and FIG. 2c can be selected depending on the size of the cover membrane 9 or the size of the bone to be regenerated, the respective anatomical situation and/or other influencing factors. In this, it is particularly advantageous that the modular design of the dental implant according to the present invention allows the two modular elements, connection element 15 on the one hand and support element 13 on the other hand, to be combined with each other as desired with regard to size etc. This means that even during a surgical operation situation, the most suitable exemplar of a connection element 15 can be selected and connected together with the most suitable exemplar of the support element 13. This ensures the best possible care.

The difference between the two support elements 13 in FIGS. 2a and 2b is the different number of cross struts 17. A higher number of cross struts 17 may result in increased stiffness against bending. However, the stiffness or bending stiffness depends on other factors, such as e.g. the cross-sectional shape of the cross struts 17 and the respective material properties. Depending on the geometric design of the support elements 13, the size of the cavity between the cover membrane 9 and the jawbone 3 can thus be influenced. This in turn has a direct influence on the regenerating jawbone and thus on the stability of the subsequent implantable dental prosthesis. The statements apply analogously for the longitudinal struts 19 and the radial struts 21 in the round embodiment shown in FIG. 2c.

One or more insertion openings 23 are arranged optionally centrally in the middle of each of the support elements 13, into which an insertion pin 25 of the connection element 15 may be inserted. The insertion pin 25 may be referred to as the first connection section 25 of the connection element 15, and the insertion opening 23 may be referred to as the second connection section 23 of the support element 13.

The length 31 of the support element 13 may be, purely exemplarily, approx. 10 mm; the width 33 may be, purely exemplarily, approx. 5 mm. Furthermore, purely exemplarily, the thickness of the cross struts 17 and/or the longitudinal struts 19 may be approx. 0.2 mm; the thickness 34 of the outer, circumferential struts may be approx. 0.25 mm. The diameter of the insertion opening 23 may be, purely exemplarily, approx. 0.8 mm. The outer diameter 35 of the round embodiment of the support element shown in FIG. 2c may be, purely exemplarily, approx. 5 mm.

FIG. 2d shows a support element 13 of a dental implant 100 according to the present invention in a side view, wherein the support element 13 may be, for example, one of the embodiments shown in FIG. 2a, FIG. 2b or FIG. 2c. The height 36 of the support element 13—which optionally remains constant at least in sections or over at least half the width—may purely exemplarily be between 0.1 mm and 0.2 mm, for example 0.16 mm.

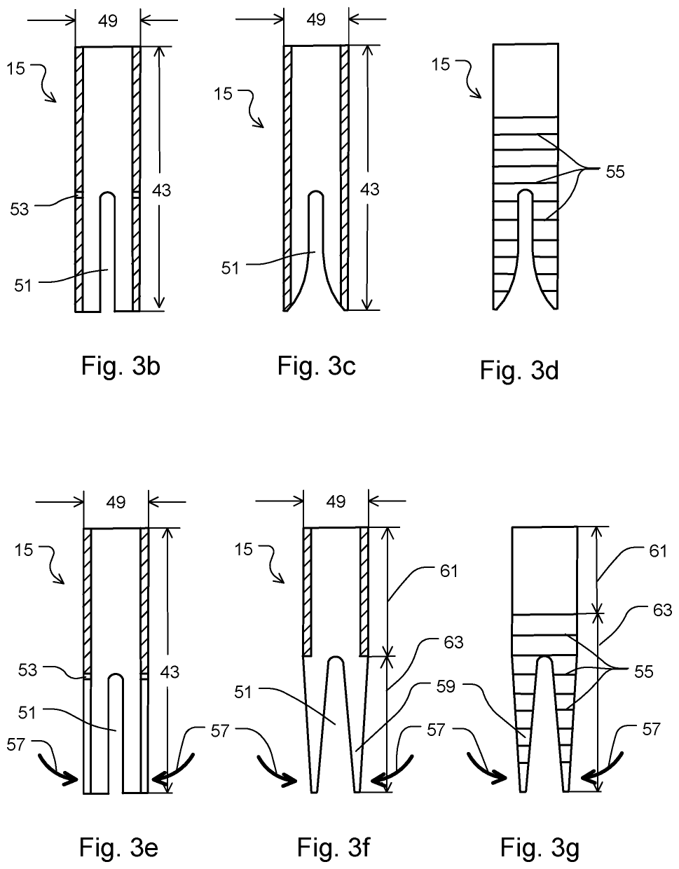

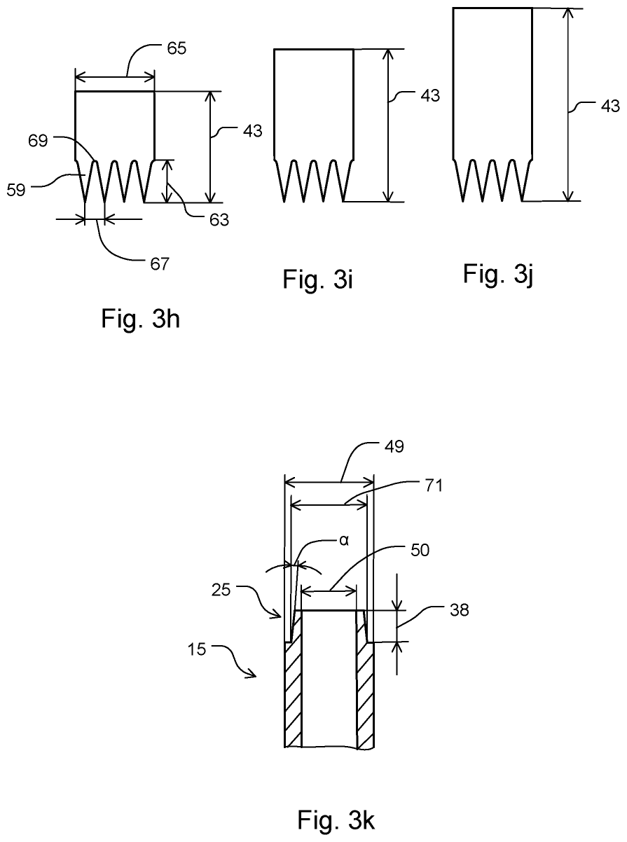

FIG. 3a shows a connection element 15 of a dental implant 100 according to the present invention having a first end section 37 and a second end section 39 for connecting the dental implant 100 to an implantation site 41 (see FIG. 5) of a jawbone 3.

The total length 43 of the connection element 15 may be, purely exemplarily, approx. 13 mm, wherein the first end section 37, which is designed as an insertion pin 25 or as a first connection section 25, has, purely exemplarily, a length 38 of approx. 0.3 mm and the second end section 39 has, purely exemplarily, a length of approx. 3 mm. The outer diameter 49 of an optionally round connection element 15 may be purely exemplarily approx. 1.2 mm.

In the enlarged view of section A of FIG. 3a, in which a section of the plugged-on or attached support element 13 is additionally shown, a cone angle α of the longitudinally-tapering first connection section 25 is shown.

This cone angle α may be manufactured or designed in the same way in the insertion opening 23 of the support element 13. Thus, the support element 13 may be pushed onto the first connection section 25 in an advantageously simple way.

Depending on the optionally selected material pairing of the first connection section 25 and the support element 13, this results in a coefficient of friction p.

The coefficient of friction p may have different values in different directions along the sliding surface, for example, depending on a structured surface. An axial direction and a tangential direction oriented perpendicular to the axial direction are given below as examples of these different directions. In the axial direction, which corresponds in particular to the direction of movement of the support element 13 relative to the connection section 25, a coefficient of friction pa may be specified in the axial direction.

The coefficient of friction p may be referred to as the friction value or friction number and is a dimensionless measure of the frictional force in relation to the contact pressure between two bodies. The friction force acts parallel to the contact surface and depends on the material-dependent and surface-dependent coefficient of friction p and a normal force acting perpendicular to the contact surface.

According to the known relationship for taper connections in machine elements, a necessary cone angle α for a self-locking taper press connection may be calculated based thereon. Accordingly, the following inequality applies to a self-locking connection between the first connection section 25 of the connection element 15 and the support element 13.

self-locking: α<arctan μₐ
α: cone angle
μₐ: coefficient of friction in axial direction
arctan: arctangent, inverse function of the trigonometric tangent function

The coefficient of friction p may be different in the axial direction and in the tangential direction, both of which are parallel to the sliding surface. Therefore, for self-locking in the axial direction, only the coefficient of friction in the axial direction is taken into account for the above inequality.

The coefficient of friction μ is determined by the material pairing and can vary in size. For example, the coefficient of static friction μ for a dry, i.e. unlubricated, material pairing of steel on steel is between approx. 0.15 and 0.3. For a value of μ=0.15, the necessary cone angle for a self-locking connection can thus be calculated as follows:

α<arctan 0.15

α<8.5° (degree) v

For a cone angle of α=5.71° chosen purely by way of example, this would therefore be a self-locking conical press connection for a material pairing steel on steel. A secure fixation or connection between the first connection section 25 of the connection element 15 and the support element 13 may thus advantageously be established.

Such a cone angle α of less than approximately 11 degrees or a cone angle α calculated according to the above formula (1) (with a specific value μ instead of 0.15 as used there by way of example) is provided in several embodiments.

FIG. 3b shows a further connection element 15 of a dental implant 100 according to the present invention in a longitudinal sectional view. In contrast to the embodiment in FIG. 3a, the further connection element 15 in FIG. 3b is tubular, hollow on the inside and designed with an optionally larger or smaller outer diameter 49 compared to the connection element 15 in FIG. 3a. This enables greater strength or bending stiffness against unintentional bending or kinking, in particular when the connection element 15 is driven into the jawbone 3.

The further connection element 15 comprises one, two or more slits 51 in the lower half, wherein if two are provided, they are circumferentially offset by 180 degrees. These slits 51 enable the lower half to be formed into an approximately pointed shape by compressing the tubular connection element 15 or by squeezing the lower end, so that an insertion or driving into the jawbone 3 is simplified.

This compression can be facilitated by—e.g. two—openings or bores 53, which are arranged offset by 180 degrees in the circumferential direction. The two bores 53 allow the connection element 15 to bend when compressed at precisely this point. In the region of these bores, plastic deformation of the material may take place in this embodiment or may be detected by finite element analysis. The compressed lower end of the further connection element 15 may thus correspond approximately to the second end section 39 of the connection element 15 of FIG. 3a.

In order to connect the further connection element 15 to a support element 13, for example a shoulder in the support element 13 may be inserted into the upper end of the tubular further connection element 15. Alternatively, a cone-shaped section may be connected to the front end as the first connection section, which can be designed analogously to the first connecting section 25 of the connection element 15 (see FIG. 3a). This is described in more detail with respect to FIG. 3h.

The total length 43 of the further connection element 15 may, purely by way of example, be between 7 mm and 15 mm, for example approx. 10 mm.

The outer diameter 49 of the further connection element 15 may be, purely by way of example, between approximately 1 mm and 2 mm, for example 1.8 mm.

The wall thickness of the further tubular connection element 15 may have, purely by way of example, a value between approx. 0.1 mm and 0.2 mm, for example 0.16 mm.

FIG. 3c shows a further embodiment of the connection element 15 in longitudinal section with pointed or tapering sections along the two slits 51. The two slits 51 (there could be more or less than two), which are offset by e.g. 180 degrees in the circumferential direction, thus widen towards the lower, front end of the connection element 15. Advantageously, this allows the two front end regions to be pressed together so that the connection element 15 can be easily driven into the jawbone.

The outer walls of the connection element 15 may be completely or substantially straight as shown in FIGS. 3b and 3c. The inner walls may extend completely, or at least partially, straight (as shown in FIG. 3b) and/or partially curved (as shown in FIG. 3c).

FIG. 3d shows the embodiment of FIG. 3c in a side view without sectional representation. Individual optional height markings 55 are shown at different heights of the connection element 15, which (all or some) can optionally be arranged around the entire circumference. These height markings 55 may advantageously be used to determine the depth of penetration into the jawbone. The height markers 55 may be arranged equidistantly or non-equidistantly. The height markings 55 may alternatively or additionally be designed as barbs to ensure firm anchoring of the connection element 15 in the jawbone. In particular with an optional absorbable material of the connection element 15, explanation is not necessary nor provided. Such height markers 55 may optionally be part of any embodiment.

FIG. 3e shows a further embodiment of the connection element 15. In contrast to the embodiments of FIGS. 3b-d, the connection elements 15 of FIGS. 3e-g comprise not two but four sections arranged over the circumference in the lower region. This is illustrated by the lower sections which are not shown in sectional representation. The four sections may be bent toward the center in the direction of arrow 57 to facilitate driving them into the jawbone.

FIG. 3f shows a further embodiment of the connection element 15, which is similar in shape to that of FIG. 3e, but comprises no openings or bores 53 and has pointed, lower sections. The lower sections may be referred to as tips 59. Compressing or plastically deforming the tips 59 toward the center advantageously enables an even more pointedly tapered shape compared to the shape of FIG. 3e, so that it is easier to drive them into the jawbone.

The upper partial length 61 and the lower partial length 63 of the connection element 15 are, purely by way of example, each approximately half as long as the total length 43.

FIG. 3g shows a side view of the connection element 15 analogous to FIG. 3d. With regard to the optional height markings 55, reference is therefore made to the description with regard to FIG. 3d.

The upper partial length 61, which represents the non-marked section of the connection element 15 in FIG. 3g, corresponds to approximately one third of the total length 43, purely by way of example. The surface of this upper section may have a different surface structure than the lower section, for example, to enable a better manual holding when driving the connection element 15 into the jawbone.

FIG. 3h, FIG. 3i and FIG. 3j show schematically simplified wind-ups of the connection element 15 shown in FIG. 3f and FIG. 3g. The length of the wind-up 65 corresponds to the circumference. With a purely exemplary outer diameter of 1.8 mm, the length of the wind-up 65 is calculated to be approx. 5.7 mm. The lower partial length 63 of the connection element 15, which in FIG. 3e corresponds to the length of the tips 59, may be between approx. 2 mm and 5 mm; in this example, the length of the tips 59 is 3 mm. The distance 67 between the tips 59 may be between approx. 1 mm and 2 mm, in this example the distance 67 is approx. 1.4 mm. The radius 69 between the tips 59 may be between approx. 0.1 mm and 0.3 mm, in this example the radius is approx. 0.2 mm.

The total length 43 of the three purely exemplary embodiments is 8 mm in FIG. 3h, 11 mm in FIGS. 3i and 14 mm in FIG. 3j. Different total lengths 43 may be advantageous if, for example, depending on the existing jawbone substance and/or depending on the size of the support element 13, it is possible to individually select between different lengths for the respective purpose of use.

FIG. 3k shows the shape of the first connecting section 25 analogous to the description with regard to FIG. 3a.

The length of the first end section 38, which corresponds to the length of the first connection section 25, may be 0.3 mm, purely by way of example.

The support element 13 can be plugged onto the conical region of the first connection section 25. Depending on the material pairing used for the connection element 15 on the one hand and the support element 13 on the other hand, the cone angle α may be selected in such a way that a self-locking conical press connection is established. The outer diameter 71 at the base of the cone, from which the cone tapers towards the end face, may be approximately 1.67 mm in this exemplary embodiment.

The reference numeral 50 denotes the inner diameter of the support element 13, which is optionally designed to be hollow or partially hollow in this embodiment.

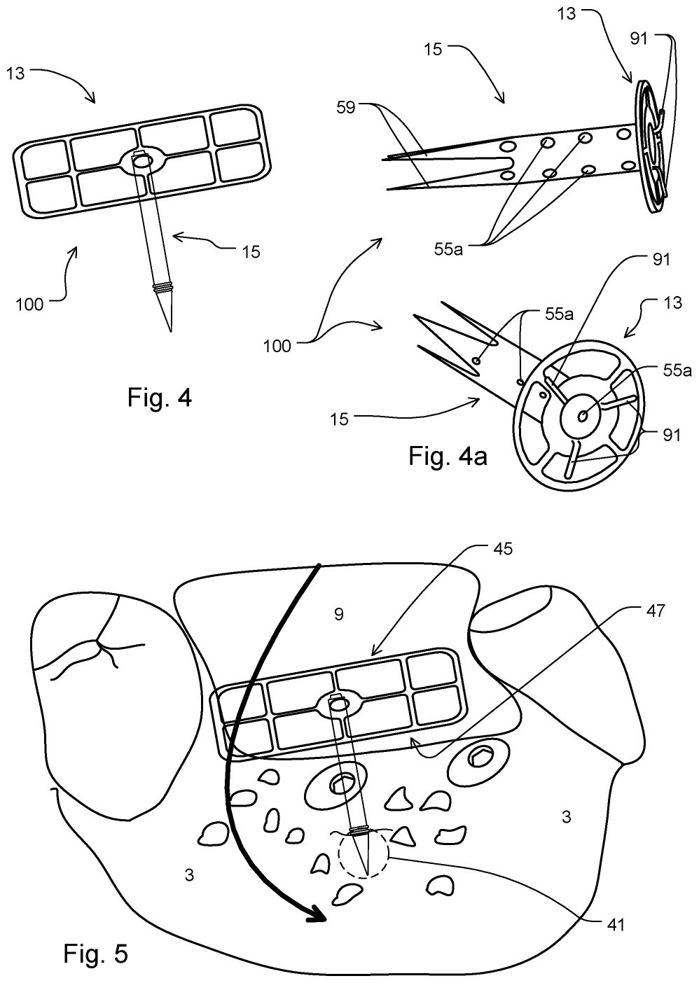

FIG. 4 shows the dental implant 100 according to the present invention in an embodiment being in the assembled state with support element 13 and connection element 15.

FIG. 4a shows a further embodiment of a dental implant 100 according to the present invention. The upper illustration of FIG. 4a is a perspective lateral view, the lower illustration of FIG. 4a is a perspective view from obliquely above.

The shape of the connection element 5 is similar in structure to the embodiment of FIGS. 3e to 3g. In this embodiment, the support element 13 is optionally round in a top view with e.g. four struts, here exemplarily oriented radially outward between an inner disk-shaped area and an outer ring of the support element 13. The protrusion of struts is optional here.

The support element 13 is fastened to the connection element 15 by three pins 91 or another number of pins 91.

The pins 91 are optionally integral components of the connection element 15, i.e. are manufactured in one piece with the connection element 15.

The production may be made, for example, from a disc-shaped or tubular semi-finished product as starting material, which is processed by laser cutting and, if necessary, later bent into the cylindrical shape shown in FIG. 4a and optionally joined together along the abutting edges in the longitudinal direction, e.g. by laser welding.

In the unassembled state of the support element 13, the pins 91 extend in the longitudinal direction optionally more or less parallel to the longitudinal axis of the support element 13.

For mounting the support element 13 on the connection element 15, these pins 91 may then be bent by about 90° (degrees) radially outwards, as shown—as a result—in FIG. 4a.

For the exact positioning of the support element 13 on the connection element 15 during assembly, the inner disk of the support element 13 may have notches for guiding the pins 91; they may run in the axial direction being perpendicular to the radial direction in which the struts extend. In the assembled state, this inner disk then rests on the connection element 15 and is fixed by the bent pins 91.

The inner disk may comprise a central opening. It may be dimensioned to fit with its edge on an optionally provided rim or shoulder, as it is disclosed in FIG. 3k. Thus, the cone—or other end face design of the connection element 15 shown in FIG. 3k may project into or through the central opening. The central opening may be designed as a blind opening or a through-opening.

The height markings 55a, in contrast to the height markings 55 of FIGS. 3d and 3g, are designed as small, round holes or openings (through-openings or blind holes). These height markings 55a may advantageously be used to determine or read the depth of penetration of the connection element 15 into the jawbone. The height markings 55a may be arranged equidistantly or non-equidistantly in the longitudinal direction. Any other design of height markings than that shown in FIG. 4a is also encompassed by the present invention.

The embodiment of the dental implant 100 shown in FIG. 4a is preferably fixed in the jawbone in the assembled state. In the assembled state, the support element 13 is already fixed to the connection element 15.

The dental implant 100 shown in FIG. 4a can be fixed in the jawbone with or without tools, for example by driving it in.

The dental implant 100 shown in FIG. 4a may also be made completely or partially of a partially or completely absorbable material.

FIG. 5 shows the bone regeneration on the jawbone 3 from FIG. 1 with the dental implant 100 according to the invention. The dental implant 100 is implanted or inserted into the jawbone 3 at the implantation site 41.

In the implantation state of the dental implant 100 shown in FIG. 5, the support element 13 has a top surface 45 facing the oral cavity of the patient and a lower surface 47 facing the implantation site 41.

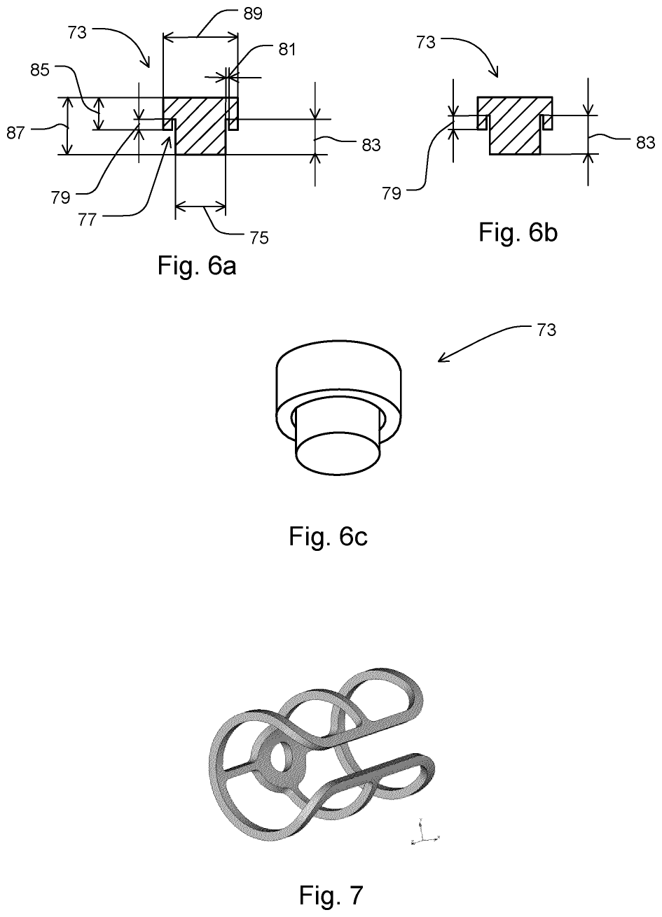

FIGS. 6a-c show two different embodiments and in different views of an exemplary tool 73 according to the present invention for driving the connection element 15 into the jawbone.

FIG. 6a shows the tool 73 in a half-sectional view in a first embodiment. The following figures are purely exemplary and may deviate therefrom in particular for different connection elements 15.

The stud diameter 75 may be selected in such that the tool 73 may be placed on the first connection section 25 of the connection element 15 with as little play as possible or, if this is designed as a hollow body at least in its upper section, may be pushed into the inner cross-section or its inner lumen (see FIG. 3k). With an exemplary outer diameter 49 of 1.8 mm and a wall thickness (in the non-conical section) of 0.16 mm, the inner diameter is 1.48 mm.

This inner diameter, which in FIG. 3k is marked with the reference numeral 50, thus corresponds to the stud diameter 75 of the tool 73, if necessary, with a fit to be selected.

The embodiment in FIG. 6a is selected in such that the first connection section 25 of the connection element 15 (see FIG. 3k) rests on the end face of the tool 73 in an optionally circumferential gap section 77 and preferably on its base (in FIG. 6a at the very top). In other words, the embodiment of the tool 73 in FIG. 6a is a tool 73 with a support for placing the tool 73 on the cone of the connection element 15.

According to the exemplary numerical example of FIG. 3k, the height 79 of the gap section 77 is 0.3 mm.

According to the exemplary numerical example in FIG. 3k, the gap width 81 corresponds to 0.95 mm and is calculated from the exemplary wall thickness of 0.16 mm minus the cone width (related to the outer diameter 71 at the cone base) of 0.65 mm. The remaining dimensions may be adjusted accordingly, thus resulting in a purely exemplary stud length 83 of 1.1 mm, an upper partial length 85 (length from the front of an upper end face to a lower end face of the gap section) of the tool 73 of 1 mm, an overall length 87 of the tool 73 from upper end face to lower end face of 1.7 mm and an outer diameter 89 of the tool 73 of 2.2 mm. The upper end face of the tool 73 with the outer diameter 89 may be used to apply the force for driving the connection element 15 into the jawbone.

In the embodiment of FIG. 6a, it should be noted that the end face of the cone of the connection element 15 rests well on the tool 73 on the end face of the gap section 77 (i.e. on the gap base). Deformation of the cone of the connection element 15 when it is driven in, may as a result be advantageously avoided, since otherwise there is the risk that the support element 13 can no longer be fitted accurately onto the cone of the connection element 15 after it has been driven in.

For example, a metal or a plastic may be used as the material for the tool, for example a polyetheretherketone (abbreviated to PEEK).

FIG. 6b shows the tool 73 in a second embodiment. In this exemplary embodiment, the height 79 of the gap section 77 is selected such that the end face of the cone of the connection element 15 does not rest on the end face of the gap section 77 (i.e. on the gap base) of the tool 73 when it is driven in. Rather, the free annular surface of the cylinder section concentrically surrounding the gap section 77, which can be seen from below in FIG. 6c, lies on the shoulder of the outer wall of the connection element 15, which can be recognized as a horizontal ring in FIG. 3K. Therefore, the height 79 of the gap section 77 may be 0.4 mm, purely exemplarily. With the same other external dimensions corresponding to the example in FIG. 6a, the exemplary stud length 83 is then 1.1 mm.

Due to the fact that—in this embodiment—the end face of the cone of the connection element 15 does not rest on the end face of the gap section 77 (i.e. on the gap bottom) when it is driven in, rather the cylinder section comes to rest on the shoulder, mechanical damage to the cone during driving in may thus be advantageously avoided.

FIG. 6c shows a perspective view of the tool 73 corresponding to the embodiments from FIGS. 6a and 6b with a view from below onto the lower end face and with a view from below into the gap section 77.

FIG. 7 shows a manufacturing variant for the support element 13. In this embodiment, the support element 13 may be manufactured from a tubular raw material. The raw material may, for example, be the same raw material as can be used to manufacture the connection element 15 according to the embodiments in FIGS. 3a to 3k. The purely exemplary outer diameter 49 of the connection element 15 has been indicated as 1.8 mm in these figure descriptions. Corresponding to the described wind-up in FIG. 3h, this corresponds to a length of approximately 5.7 mm. Having an exemplary width 33 of the support element 13 of 5 mm, makes it sufficient for manufacturing the support element 13. Production may take place, for example, by laser cutting. The basic method for an absorbable material such as a magnesium alloy is known from the manufacture of stents from medical technology applications. However, the support element must be plastically deformed or expanded after manufacture.

### LIST OF REFERENCE NUMERALS

100 dental implant
α cone angle of the first connection section
μₐ coefficient of friction in the axial direction
1 bone replacement material; granules
3 jawbone
5 tooth
7 screw; placeholder
9 cover membrane; cover film; cover element
11 arrow direction for moving the cover membrane
13 support element
15 connection element
17 cross strut
19 longitudinal strut
21 radial strut
23 insertion opening; second connection section
25 insertion pin; first connection section
31 length of the support element
32 thickness of the cross strut and/or of the longitudinal strut
33 width of the support element
34 thickness of the outer, circumferential or closed strut
35 outer diameter of the round support element
36 height of the support element
37 first end section of the connection element
38 length of the first end section
39 second end section of the connection element
41 implantation site
43 total length of the connection element
45 top surface of the support element
47 lower surface of the support element
49 outer diameter of the connection element
50 inner diameter
51 slit
53 bore
55,55a height markings of the connection elements
57 arrow direction
59 tips
61 upper partial length of the connection element
63 lower partial length of the connection element
65 length of the wind-up; circumference
67 distance between the tips
69 radius between the tips
71 outer diameter at the base of the cone
73 tool for driving in the connection element
75 stud diameter
77 gab section
79 height of the gab section
81 width of gab
83 length of stud
85 upper partial length of the tool
87 total length of the tool
89 outer diameter of the tool
91 pin

1. A dental implant for supporting, in particular by using a support structure, a cover element, comprising
a connection element with
a first end section and with
a second end section for connecting the dental implant to a jawbone; and
a first support element for supporting a cover element after implantation of the dental implant within the jawbone;
wherein the first end section is embodied as, or comprises, a first connection section;
wherein the first support element comprises a second connection section;
wherein the first connection section and the second connection section are embodied to be interconnectable or connectable to each other;
wherein the second end section embodied as, or comprises, an implantation section for temporarily implanting the connection section on or in the implantation site of the jawbone.

2. The dental implant according to claim 1, wherein the first and the second connection sections correspond to each other, wherein the first connection section or the second connection section is preferably embodied as an insertion opening, and wherein the other of these two connection sections is embodied as an insertion pin.

3. The dental implant according to claim 1, wherein the second end section is embodied as a pointed or tapered or tapering section.

4. The dental implant according to claim 1, wherein the first connection section and the second connection section are embodied for releasable connection to each other.

5. The dental implant according to claim 1, wherein the first support element is, or comprises, a grid-shaped support structure.

6. The dental implant according to claim 1, wherein the first support element comprises a top surface facing the oral cavity of the patient in the implantation state of the dental implant and a lower surface facing the implantation site, wherein the second connection section is embodied to receive the first connection section at said lower surface.

7. The dental implant according to claim 1, wherein the connection element does not comprise a threaded section and/or does not comprise a screw head.

8. The dental implant according to claim 1, wherein the connection element and/or the first support element is made of, or comprises, a metal, a plastic and/or a composite material.

9. The dental implant according to claim 1, wherein the connection element and/or the first support element is made entirely or partially of an absorbable material or comprises such a material.

10. A set, encompassing a dental implant according to claim 1, further comprising
at least one further support element, wherein the first support element and the second support element differ in at least one geometric feature, and/or
an implantable dental prosthesis.

11. The set according to claim 10, wherein the geometric feature encompasses the shape, a dimension, the surface and/or the arrangement of support structure elements.

12. A tool for inserting a connection element of a dental implant according to claim 1 into a bone, wherein the tool comprises a gap section for receiving an end section of the connection element.
